(19) United States
(12) Patent Application Publication
(10) Pub. No.: US 2024/0238580 A1
(43) Pub. Date: Jul. 18, 2024

(54) OUTFLOW TUBE AND OUTFLOW TUBE ASSEMBLIES

(71) Applicant: ABIOMED, Inc., Danvers, MA (US)

(72) Inventor: Keith Swenson, Danvers, MA (US)

(73) Assignee: ABIOMED, Inc., Danvers, MA (US)

(21) Appl. No.: 18/411,256

(22) Filed: Jan. 12, 2024

Related U.S. Application Data
(60) Provisional application No. 63/438,881, filed on Jan. 13, 2023.

(51) Int. Cl. A61M 60/13; A61M 60/232; A61M 60/81; A61M 60/859

(52) U.S. Cl. CPC A61M 60/13; A61M 60/232; A61M 60/81; A61M 60/859; A61M 2205/103; A61M 2205/3331; A61M 2205/75; A61M 2207/00

(57) ABSTRACT

A one-piece outflow tube balloon, either alone or as part of another device, and a method of manufacturing the device, may be provided. The balloon may include a flexible tubular polymeric layer, having a distal end and a proximal end, and having an outer surface and an inner surface. The balloon may include one or more openings at a proximal end, the one or more openings extending from the outer surface to the inner surface. The balloon may include a slot extending from the proximal end to a proximal end of one of the one or more openings. The balloon may include a distal end configured to be disposed over an expandable housing.

### CROSS-REFERENCE TO RELATED APPLICATIONS

The present application claims priority to U.S. 63/438,881, filed Jan. 13, 2023, the contents of which are incorporated by reference herein in its entirety.

### TECHNICAL FIELD

The present disclosure is drawn to outflow tubes for catheter-based heart pumps.

### BACKGROUND

With catheter-based heart pumps, outflow tubes may be used to control the flow of blood after the blood has left the pump housing. However, the design of such outflow tubes means the manufacturing process to produce such outflow tubes and affix them to the heart pumps is complex. Such complexity may lead to defects.

### BRIEF SUMMARY

The present system and techniques improve upon the prior art in various ways.

In some embodiments, a one-piece outflow tube balloon may be provided. The one-piece outflow tube balloon may include one or more openings at a proximal end of the balloon. The balloon may include a slot extending from the proximal end of the balloon to a proximal end of one of the one or more openings. The balloon may include a middle portion configured to be operably coupled to an expandable housing. The balloon may include a distal portion configured as an inflow mesh.

In some embodiments, an outflow tube assembly may be provided, where the outflow tube assembly may include a one-piece outflow tube balloon as disclosed herein operably coupled to an expandable housing and/or an inner coating of the housing. The housing may include a filter portion at the inflow.

In some embodiments, a medical device may be provided, where the medical device may include a one-piece outflow tube balloon as disclosed herein operably coupled to a catheter at a proximal end and operably coupled an expandable housing and/or an inner coating of the housing.

In some embodiments, a method for manufacturing a device, where the method may include creating a first subassembly by providing an inner coating onto a mandrel, creating a second subassembly by providing an expandable housing over the inner coating, creating a third subassembly by disposing a one-piece outflow tube balloon over the housing and inner coating, and coupling the inner coating and housing to a portion of the one-piece outflow tube balloon (via, e.g., thermal welding), and optionally, removing a portion of the one-piece outflow tube balloon to form an inflow mesh.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of an outflow tube assembly.

FIG. 2 is an illustration of an inner coating.

FIG. 3 is an illustration of a housing.

FIG. 4 is an illustration of an outflow tube balloon.

FIG. 5 is a cross-sectional view of the inflow region of the expandable housing of FIG. 4, with an expandable filter installed thereon, and also illustrating an inner coating.

FIG. 6 is a side view of a housing, outflow tube, and an expandable filter.

FIG. 7 is a perspective view of the inflow region of the expandable housing of FIG. 6.

FIG. 8 is a flowchart of a manufacturing process for an outflow tube.

FIG. 9A is a front view of an embodiment of an outflow tube balloon.

FIG. 9B is a back view of an embodiment of an outflow tube balloon.

FIG. 9C is a top view of an embodiment of an outflow tube balloon.

FIG. 9D is a bottom view of an embodiment of an outflow tube balloon.

FIG. 9E is a right view of an embodiment of an outflow tube balloon.

FIG. 9F is a left view of an embodiment of an outflow tube.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

An improved outflow tube may be provided, whereby various elements or manufacturing steps can be avoided. For example, in various embodiments, two thermo-welding transformations may be avoided, an outer coating may no longer be needed, four processing aids (e.g., various mandrels or other tools used during manufacturing) are no longer necessary, and there is no need to cut away excess outflow tube during or after manufacturing.

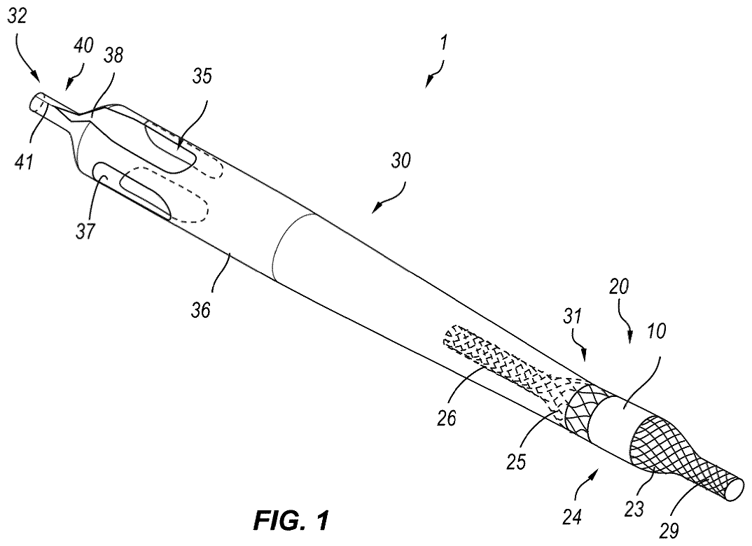

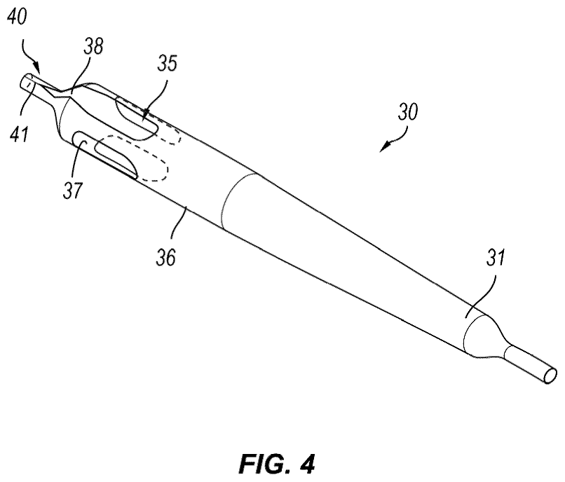

Referring to FIG. 1, an outflow tube assembly can be seen. The outflow tube assembly 1 may include an inner coating 10. The inner coating may be disposed on an inner surface of an expandable housing 20. Note, in FIG. 1, the expandable housing around the inner coating is not shown for ease of understanding. FIG. 3 shows an example of a typical expandable housing.

Because these devices are configured to be placed into, e.g., blood vessels of a patient, the housing may have an inflow 23 configured to allow blood to enter the housing, an outflow 25 configured to allow blood to exit the housing, and may have a middle portion 24 extending between a proximal end of the inflow and a distal end of the outflow. The housing may have a proximal portion 26 extending proximally from a proximal end of the outflow. The proximal portion may be configured to be coupled to a catheter (not shown). The housing may have a distal portion 29 extending distally from a distal end of the inflow.

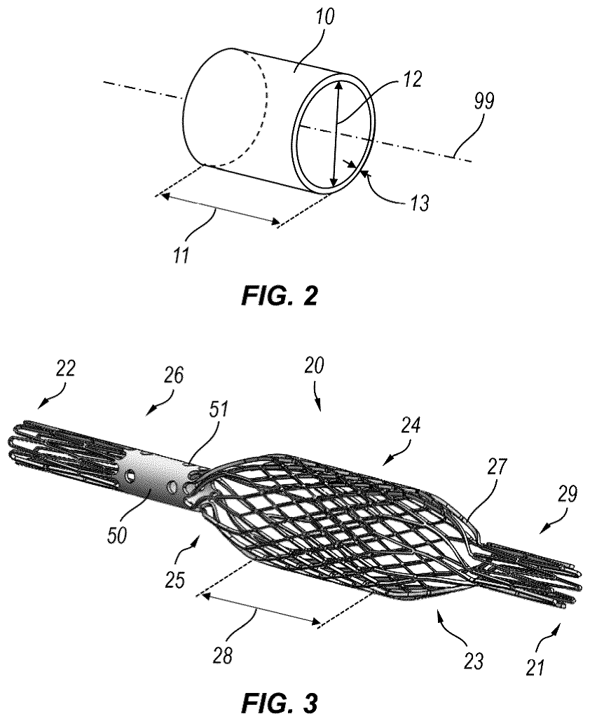

Simplified embodiments of these two elements (inner coating and expandable housing) can be seen FIGS. 2 and 3.

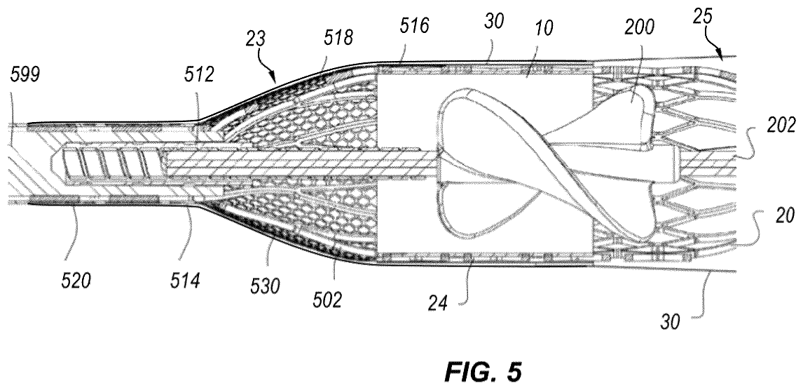

The inner coating 10 is shown in FIG. 2 as being generally cylindrical around a central axis 99, having an axial length 11, an inner diameter 12, and a thickness 13. In some embodiments, the axial length may be equal to an axial length of the middle portion 24 of the housing. That is, in some embodiments, the axial length 11 may be equal to an axial distance 28 between the proximal end of the inflow a distal end of the outflow (see FIG. 4A). In some embodiments, the axial length may be less than an axial length of the middle portion. The inner diameter is generally smooth, to reduce any effect on the flow of blood through the middle portion.

An embodiment of the expandable housing 20 is show in FIG. 3. The housing 20 may have a distal end 21 and a proximal end 22. The housing may be formed at least partially from a plurality of struts 27, the struts being configured to form an inflow 24, an outflow 26, and a middle portion extending between a proximal end of the inflow and a distal end of the outflow. The middle portion may have an axial length 28. The housing may include a proximal portion 26 formed proximal to the outflow, which may be formed from the struts. The housing may include a distal portion 29 formed distal to the inflow, which may be formed from the struts.

The housing may be radially expandable. The inflow, middle portion, and outflow of the housing may be configured to have a compressed state and an expanded state. The housing may be configured to be disposed around a catheter (not shown).

If the housing is expandable, the housing may include a plurality of struts, represented by struts made of a suitable shape memory, hyperelastic or superelastic material, such as Nitinol. Hyperelastic materials are typically elastomers. Many such elastomers can elastically deform up to about 100%. Some superelastic materials can elastically deform up to about 6-8%. Nitinol is a trade name for a nickel-titanium alloy distinguished from other materials by its shape memory and superelastic characteristics.

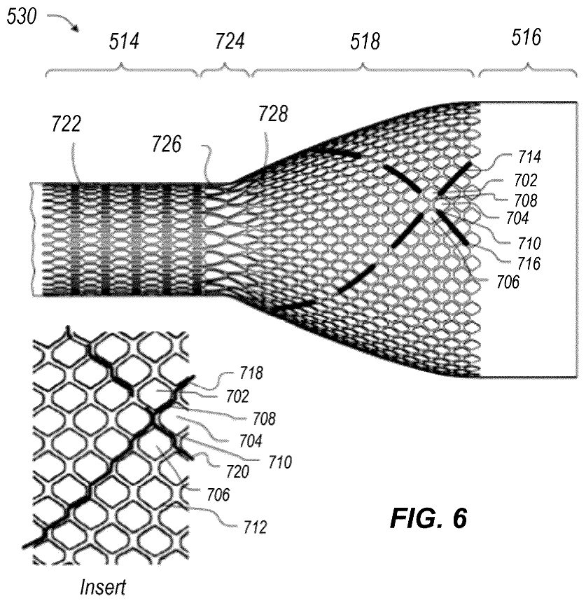

The struts may be made of wire or other filament. The housing is generally configured to provide a “cage” around an impeller positioned within the housing (see impeller 200 in FIG. 5). When radially expanded, the length of the housing may be less than the length when the housing is radially compressed. The change in length to may be due to unwinding of the struts, when the housing expands. In some embodiments, the change in length when changing from compressed to expanded state may be about 1-2 mm.

In some embodiments, the proximal portion may include a connector 50, which may be a rigid connector coupled to one or more struts. The connector may define a lumen (not shown) extending from a proximal to a distal end of the connector. The connector may be configured with one or more openings 51 extending from an outer surface to an inner surface of the connector. The connector may be configured to be coupled to a catheter (not shown).

The outflow tube assembly 1 may include an outflow tube balloon 30 coupled, where at least a portion 31 is configured to be operably coupled to the housing and/or the inner coating. The outflow tube balloon may be flexible. In some embodiments, the outflow tube balloon may include a polymer, such as PET or PU. The thickness of the outflow tube balloon may be, e.g., about 10 μm to about 100 μm thick. The thickness of the outflow tube balloon may vary in an axial direction. The outflow tube balloon may be formed from using, e.g., a blow mold manufacturing process.

The outflow tube balloon may include one or more openings 35 extending from an outer surface 36 of the outflow tube balloon to an inner surface 37. In some embodiments, the one or more openings may have an identical shape. In some embodiments, one or more of openings may have a different shape. The outflow tube may be configured to be coupled to a catheter (not shown) at a proximal end 32, positioned similar to the position of the mandrel.

The outflow tube balloon is configured to be disposed around and proximal to the outflow from the housing. In this manner, blood flowing out of the outflow will enter a volume of space defined by the inner surface of the outflow tube balloon, and blood will flow in a generally axial direction towards the one or more openings, where the blood will then exit the outflow tube and be passed back into a blood vessel.

To aid in assembly and manufacturing, the outflow tube may include a slot 40 or slit extending through a portion of the outflow tube balloon, from a proximal end to one of the one or more openings 35. In some embodiments, the slot may include a straight portion 41 extending axially from the proximal end towards a triangular-shaped portion 38 of the one or more openings 35, where the apex of the triangular-shaped portion connects to the slit.

The one or more openings may include a plurality of openings. In some embodiments, each opening is an identical circumferential distance from an adjacent opening. In some embodiments, at least a first opening is a different circumferential distance from an adjacent opening as compared to a second opening (e.g., the openings may not be identically spaced around a circumference of the outflow tube balloon). In some embodiments, there may be 3-8 openings. In some embodiments, there may be 3-4 openings. In some embodiments, there may be 4 openings.

In FIG. 5, use of the disclosed outflow tube may be seen in conjunction with a medical device. The medical device may be, e.g., a blood pump, such as a catheter-based blood pump.

An impeller 200 is shown located inside the housing 20 and mechanically coupled via the flexible drive shaft 202 to a proximally-located motor (not shown).

An inside central portion of the housing 20 may have an inner coating 10, which defines a channel through which the blood is pumped by the impeller 200. Proximally and distally of this channel, the housing 20 allows blood to be sucked into the housing through the inflow 23 and pushed out of the housing through the outflow 25 into a downstream portion of the outflow tube balloon 30. The housing may include a proximal tapered housing part 502 adjacent to intermediate housing part 24.

In some embodiments, the housing may include an inflow mesh or filter portion. In some embodiments, the housing may include a portion disposed on the outside of the housing, forming an expandable filter 530. In some embodiments, the filter 530 may include a distal tubular filter section 514, which has a relatively small diameter, and a proximal tubular filter section 516, which as a larger diameter (in its expanded state). The exact cross-sectional shape of the filter 530, including the exact cross-sectional shape of the distal tubular filter section 514 and the proximal tubular filter section 516, may depend on a number of struts in the housing and/or in the filter. In general, the cross-sectional shape may be a polygon, possibly with rounded corners. The distal tubular filter section 514 may be disposed on top of the distal bearing 512.

In some embodiments, a distal end of the outflow tube balloon may be heat sealed, such as by welding, through one or more holes defined by the struts of the distal tubular filter section 514, extending to a proximal section of the flexible atraumatic tip 599.

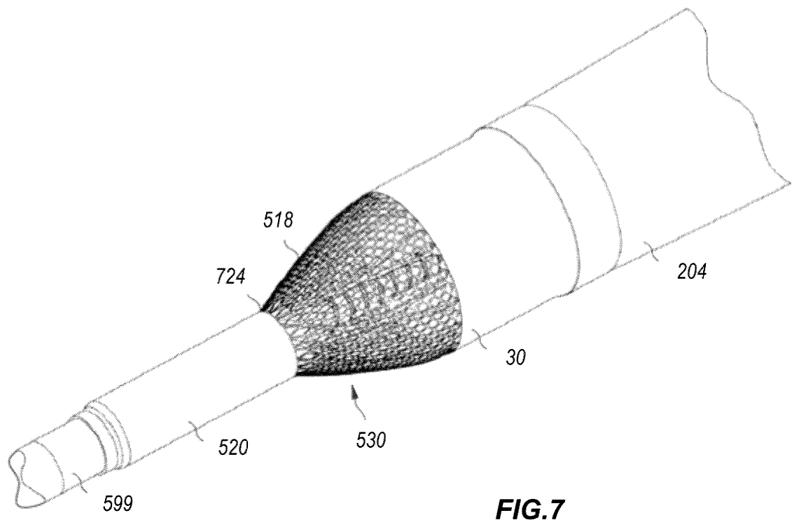

As seen in FIG. 6, in some embodiments, the expandable filter 530 may include a transitional zone 724 where the distal tubular filter section 514 and the tapered filter section 518 meet. Holes, exemplified by hole 726, in the transitional zone 724 are longer and wider than adjacent holes of the tapered filter section 518. Preferably, the holes 726 in the transitional zone 724 are at least twice as large as the adjacent holes, exemplified by hole 728, in the tapered filter section 518. In one embodiment, for each pair of circumferentially adjacent holes 728 in a row of the tapered filter section 518, the transitional zone 724 has one hole 726 that circumferentially straddles the two holes 728. Thus, the number of holes in a circumferential row in the transitional zone 724 is half the number of holes in a circumferential row in the tapered filter section 518. In some other embodiments, other ratios may be used, such as 3:1, 4:1 or 3:2. Each hole 726 in the transitional zone 724 may be about twice, thrice or another multiple as long (in the longitudinal direction) and about twice, thrice or another multiple as wide (in the circumferential direction) as the hole 728 in the tapered filter section 518, depending on the ratio of the number of holes 728 in one row of the tapered filter section 518 to the number of holes 726 in one row of the transitional zone 724.

The dimensions and shapes of the holes 702-706 and 728 and dimensions of the struts 714-716 should be chosen such that, when the tapered filter section 518 is fully open, the inflow of the housing in its expanded state can be disposed within the tapered filter section 518, without exceeding limits of elastic deformation of the material. For example, the length of two circumferentially adjacent struts 714-716 (on zigzag of a zigzag circumferential ring), multiplied by the number of apertures 702-706 in a circumferential row, should about equal the circumference of a fully-expanded housing, taking into account any local elastic deformation of the filter material.

The apertures 702-706 are positioned, such that material, exemplified by material 708, 710 and 712, between the apertures 702-706 forms first and second struts. Two exemplary struts 714 and 716 are indicated in FIG. 6 by heavy dashed lines. As noted, a generally helical curve may include minor zigzags, not necessarily all the same, as exemplified by generally helical curves 714 and 716. These zigzags are more clearly seen in the insert in FIG. 6, for example in struts 718 and 720, which are indicated by heavy solid and dashed lines.

Adjacent holes 726 in the transitional zone 724 are separated from each other by struts that are wider than an adjacent strut 714-716 of the tapered filter section 518. These wider struts stabilize the larger holes 726. When the distal portion 520 of the outflow tube balloon is placed over of the distal tubular filter section 514, longitudinally proximally up to the transitional zone 724, the distal portion 520 at least partially covers, and therefore reduces the effective size of, the first one or more rows of the holes 726 in the transitional zone 724. In some cases, these reduced hole sizes may lead to blood damage or increased risk of clotting. Therefore, the holes 726 in the transitional zone 724 should be chosen to be larger than holes in the tapered filter section 518.

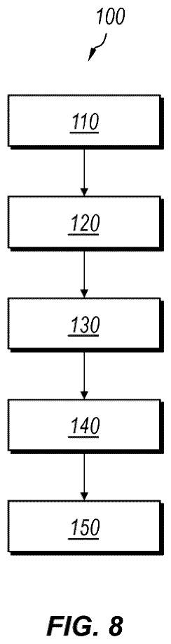

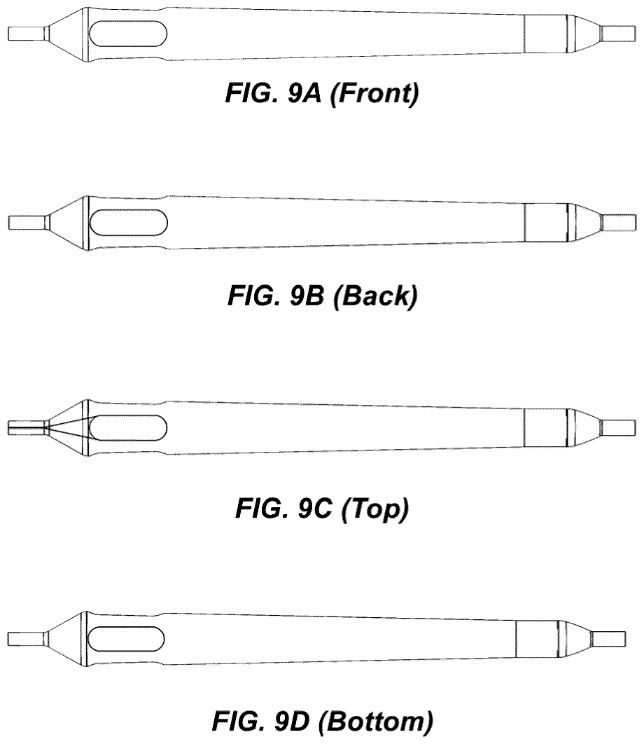

As can be seen in FIGS. 6 and 7, the holes 728 in a distal region of the tapered filter section 518 are narrower, in a circumferential direction, than the holes 702-706 in a proximal region of the tapered filter section 518. In other words, sizes of the apertures 702-706 increase monotonically in the proximal direction, along the longitudinal axis. In addition, in the distal tubular filter section 514, the holes 722 take the form of narrow axial slits, which are offset from each other in a circumferential direction. This is advantageous, as narrow holes can widen when the expandable filter 530 is expanded at the distal tubular filter section 514 and the distal region of the tapered filter section 518, such as when the impeller 200 is inserted into the housing. Wider holes are bounded by thicker struts, particularly in the tapered filter section 518. The struts have a width of between about 30 μm in the distal region, and about 60 μm in the proximal region, of the tapered filter section 518. Preferably, the largest diameter of the holes in the tapered filter section 518 is between about 300 μm and about 500 μm.

In the embodiment shown in FIGS. 6 and 7, the proximal tubular filter portion 516 has no holes. However, holes in the proximal tubular filter portion 516 may be desirable, such as when the one-piece outflow tube balloon 30 is placed over the proximal tubular filter section 516 (see FIG. 5), which in turn is situated over the middle/intermediate housing part 24.

As such, in some embodiments, the portion of the outflow tube disposed around the inflow may include a plurality of apertures aligned with the apertures of the inflow 23 and/or the filter 530.

A method for manufacturing such devices can be seen with reference to FIG. 2. The method 100 may include a first step of assembling 110 an inner coating onto a mandrel, forming a first subassembly.

The method may include a second step of assembling 120 a housing onto the first subassembly, forming a second subassembly.

The method may include inserting 130 the outflow tube balloon, where a distal end of the mandrel is passed into a proximal end of the outflow tube balloon, the outflow tube balloon is then brought proximally over the second subassembly, until the outflow tube balloon is placed correctly, forming an third subassembly.

The method may include a step 140 of coupling the outflow tube balloon to the housing and/or inner coating, and/or ablating the outflow tube balloon. Specifically, the outflow tube balloon may be coupled to the housing and/or inner coating via an appropriate method, such as thermal bonding. The outflow tube balloon may be ablated via, e.g., laser ablation, to cut a design in at least a portion of the outflow tube balloon. This step forms the outflow assembly.

The method may include removing 150 the mandrel from the outflow assembly.

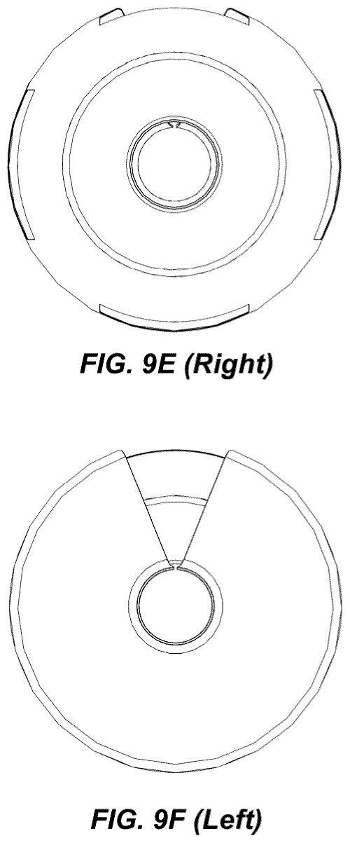

In some embodiments, various views of an embodiment of an outflow tube balloon can be seen in FIGS. 9A-9F, prior to any laser ablation.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed:

1. A one-piece outflow tube balloon, comprising:
a flexible tubular polymeric layer, having a distal end and a proximal end, and having an outer surface and an inner surface;
one or more openings at a proximal end, the one or more openings extending from the outer surface to the inner surface;
a slot extending from the proximal end to a proximal end of one of the one or more openings; and
a distal end configured to be disposed over an expandable housing.

2. The one-piece outflow tube balloon of claim 1, wherein the distal end is configured as an inflow mesh.

3. An outflow tube assembly, comprising:
an expandable housing defining an inflow and an outflow; and
an inner coating disposed against an inner surface of the expandable housing between the inflow and the outflow; and
a one-piece outflow tube balloon according to claim 1, a portion of the one-piece outflow tube balloon operably coupled to the expandable housing and/or the inner coating.

4. The outflow tube assembly according to claim 3, wherein the housing includes a filter portion at the inflow.

5. A medical device, comprising:
a flexible tubular member; and
an outflow tube assembly according to claim 3, the outflow tube assembly having a distal end and a proximal end, where the distal end and proximal end are operably coupled to the flexible tubular member.

6. The medical device according to claim 5, further comprising an impeller disposed within the outflow tube assembly.

7. A method of manufacturing a device, comprising:
creating a first subassembly by providing an inner coating onto a mandrel;
creating a second subassembly by providing an expandable housing over the inner coating;
creating a third subassembly by disposing a one-piece outflow tube balloon over the housing and inner coating;
forming the device by coupling the inner coating and housing to a portion of the one-piece outflow tube balloon (via, e.g., thermal welding), and optionally, removing a portion of the one-piece outflow tube balloon to form an inflow mesh; and
removing the device from the mandrel.
